(19) United States
(12) Patent Application Publication
(10) Pub. No.: US 2007/0122355 A1
(43) Pub. Date: May 31, 2007

(54) RAPIDLY ABSORBING ORAL FORMULATIONS OF PDE 5 INHIBITORS

(75) Inventors: David Monteith, Pittstown, NJ (US); Pascal Borderies, New York, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/615,548

(22) Filed: Dec. 22, 2006

Related U.S. Application Data
(62) Division of application No. 11/472,628, filed on Jun. 22, 2006.
(60) Provisional application No. 60/693,219, filed on Jun. 23, 2005.

(51) Int. Cl. A61K 9/68; A61K 31/519; A61K 9/20

(52) U.S. Cl. 424/48; 424/464; 514/252.16; 514/262.1

(57) ABSTRACT

The present invention encompasses oral formulations of a PDE5 inhibitor which provide rapid disintegration after introduction to the oral cavity, followed by buccal and/or sublingual absorption. The orally disintegrating formulations can be in a variety of dosage forms including lingual strip, sublingual strip, oral mist, rapidly disintegrating tablet, lyophilized wafer, granulated particles and gum. The formulations can include an extended release component that allows the PDE5 inhibitor to be swallowed for gastrointestinal absorption. Combination therapies with a second pharmaceutical agent known to cause a PDE5-treatable condition as a side effect, such as erectile dysfunction, are also described.
The PDE5 inhibitor of the following chemical structure is particularly favored for these formulations:

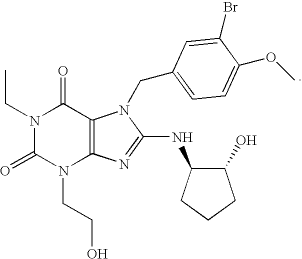

### CROSS REFERENCE TO RELATED APPLICATIONS

This application claims the benefit of U.S. Provisional Application no. 60/693,219, filed on Jun. 23, 2005.

### FIELD OF THE INVENTION

This invention encompasses orally disintegrating pharmaceutical formulations for the rapid absorption and onset of action of phosphodiesterase 5 (PDE5) inhibitors. The invention also encompasses the use of the pharmaceutical formulations of PDE5 inhibitors for treating diseases beneficially affected by such PDE5 inhibitors. In particular, the invention encompasses the buccal and/or sublingual administration of at least one PDE5 inhibitor.

### BACKGROUND OF THE INVENTION

A wide variety of biological processes, including cardiac muscle contraction, regulation of blood flow, neural transmission, glandular secretion, cell differentiation and gene expression are affected by steady state levels of the cyclic nucleotide biological second messengers cAMP and cGMP. Intracellular receptors for these molecules include cyclic nucleotide dependent protein kinases (PGK), cyclic nucleotide-gated channels, and class I phosphodiesterases (PDEs). PDEs are a large family of proteins, which were first reported by Sutherland and co-workers (Rall & Sutherland 1958, Butcher & Sutherland 1962). The family of cyclic nucleotide phosphodiesterases catalyzes the hydrolysis of 3′, 5′-cyclic nucleotides to the corresponding 5′ monophosphates. Literature shows that there are eleven related, but biochemically distinct, human phosphodiesterase gene groups and that many of these groups include more than one oene subtype giving a total of twenty genes.

Some PDEs are highly specific for hydlolysis of cAMP (PDE4, PDE7, PDE8), some are highly cGMP specific (PDE5, PDE6, PDE9), and some have mixed specificity (PDE1, PDE2, PDE3, PDE10, PDE11). All PDEs are multi-domain proteins; each PDE has about 270 amino acid domains located towards the C-terminus, which has a high degree of amino acid sequence conservation between families. This domain has been extensively studied and shown to be responsible for the common catalytic function. Non-homologous segments in the remainder of the protein have regulatory function or confer specific binding properties. PDE2, PDE5, PDE6 and PDE10 are all reported to contain putative GAF domains within their regulatory amino terminal portion. These GAF domains have been shown to bind cGMP but their function is not yet fully understood. Full length mammalian PDEs characterized to date are dimeric in solution, but the relevance of the dimeric structure is unknown.

The PDE5 receptor, a cGMP specific PDE receptor, has been recognized in recent years as an important therapeutic target. It is composed of the conserved C-terminal, zinc containing, catalytic domain, which catalyses the cleavage of cGMp, and an N-terminal regulatory portion, which contains two GAF domain repeats. Each GAF domain contains a cGMP-binding site, one of high affinity and the other of lower affinity. PDE5 activity is regulated through binding of cGMP to the high and low affinity cGMP binding sites followed by phosphorylation, which occurs only when both sites are occupied. PDE5 receptors are found in varying concentrations in a number of tissues including platelets, vascular and visceral smooth muscle, and skeletal muscle. The protein is a key regulator of cGMP levels in the smooth muscle of the erectile corpus cavernosal tissue.

The physiological mechanism of erection involves release of nitric oxide (NO) in the corpus cavernosum during sexual stimulation. NO then activates the enzyme guanylate cyclase, which results in increased levels of cGMP, producing smooth muscle relaxation in the corpus cavernosum and allowing inflow of blood. Inhibition of PDE5 receptors inhibits the breakdown of cGMP, allowing the levels of cGMP, and the consequent smooth muscle relaxation, to be maintained. For example, sildenafil, the active ingredient of Viagra® and a potent inhibitor of PDE5 receptors, has attracted widespread attention for the effective treatment of male erectile dysfunction.

A number of causes of impotence have been identified, including vasculogenic, neurogenic, endocrinologic and psychogenic. Vasculogenic impotence, which is caused by alterations in the flow of blood to and from the penis, is thought to be the most frequent organic cause of impotence. Common risk factors for vasculogenic impotence include hypertension, diabetes, cigarette smoking, pelvic trauma, and the like. Neurogenic impotence is associated with spiral-cord injury, multiple sclerosis, peripheral neuropathy caused by diabetes or alcoholism and severance of the autonomic nerve supply to the penis consequent to prostate surgery. Erectile dysfunction is also associated with disturbances in endocrine function resulting in low circulating testosterone levels and elevated prolactin levels.

Impotence can also be a side effect of various classes of drugs, in particular, those that interfere with central neuroendocrine control or local neurovascular control of penile smooth muscle. Krane et al., New England Journal of Medicine 32:1648 (1989). Penile erection requires, (1) dilation of the arteries that regulate blood flow to the lacunae of the corpora cavemosum; (2) relaxation of trabecular smooth muscle, which facilitates engorgement of the penis with blood; and, (3) compression of the venules by the expanding trabecular walls to decrease venous outflow.

The formulations of the prior art fail to provide a rapid onset of action of PDE5 inhibitors and use large dosages, because the PDE5 inhibitor is administered via conventional oral formulations that are absorbed gastrointestinally.

### SUMMARY OF THE INVENTION

The invention encompasses a pharmaceutical formulation comprising a rapid release component comprising at least one PDE5 inhibitor and an orally disintegrating carrier, wherein the rapid release component results in a therapeutically effective blood concentration of the PDE5 inhibitor in about 1 minute to about 20 minutes. In some embodiments, this concentration is attained in less than about 10 minutes, and in other embodiments, this concentration is attained in less than about 5 minutes.

In some embodiments, the rapid release component disintegrates within about 1 second to about 10 seconds. In some embodiments, the rapid release component disintegrates in less than about 5 seconds.

In some embodiments the PDE5 is selected from the group consisting of SCH446132, sildenafil citrate, tadalafil, vardenafil, avanafil and udenafil.

In some embodiments, the pharmaceutical formulation is in a dosage form selected from the group consisting of lingual strips, sublingual strips, oral mists, rapidly disintegrating tablets, lyophilized wafers, granulated particles and gums. In some embodiments, the PDE5 inhibitor is present in an amount of about 3 mg.

In some embodiments, the formulation results in a therapeutically effective blood concentration of the PDE5 inhibitor in about 3 minutes or less. In some embodiments, the formulation results in a Cₘₐₓ of about 5 μ/L to about 60 μg/L in about 5 minutes to about 10 minutes. In some emnbodiments, the formulation results in an AUC of about 10 μgh/L to about 200 μgh/L.

In some embodiments, the pharmaceutical formulation further comprises at least one permeation enhancer selected from the group consisting of DMSO, DMF, DMA, C₁₀ MSO, PEGML, glycerol monolaurate, lecithin, 1-substituted azacycloheptan-2-ones, alcohols, and surfactants.

In some embodiments, the pharmaceutical formulation comprises SCH446132 in a lyophilized lingual/sublingual wafer. In some embodiments, the pharmaceutical formulation comprises SCH446132 and an effervescent agent.

In some embodiments, the pharmaceutical formulation comprises SCH446132 in a spray mist.

In some embodiments, the pharmaceutical composition further comprises an extended release component comprising at least one PDE5 inhibitor and a non-orally disintegrating carrier. In some embodiments, the pharmaceutical formulation is formed as a tablet comprising a core comprising the extended release component and a coating comprising the rapid release component. In some embodiments, the pharmaceutical formulation is formed as a strip and the extended release component comprises granulated particles. In some embodiments, the formulation results in an AUC of about 20 μgh/L to about 400 μgh/L.

In some embodiments, the pharmaceutical formulation further comprises at least one second pharmaceutical agent. In some embodiments, the second pharmaceutical agent is selected from those known to cause a PDE5-treatable condition. In some embodiments, the PDE5-treatable condition is erectile dysfunction. In some embodiments, the PDE5-treatable condition is premature ejaculation. In some embodiments, the second pharmaceutical agent is an SSRI. In some embodiments, the second pharmnaceutical agent is paroxetine. In some embodiments, the second pharmaceutical agent is selected from those known to treat craniopharyngioma, diabetes, epilepsy, hypogonadism, hypertension, ischemic heart disease, multiple sclerosis, and/or a peripheral vascular disease.

### DETAILED DESCRIPTION OF THE INVENTION

The methods and formulations of the present invention effectively deliver a PDE5 inhibitor in a rapidly orally disintegrating formulation that provides substantial buccal and/or sublingual absorption in a manner to rapidly achieve effective blood levels at a lower dosage than required in the prior art.

Orally absorbed agents are relatively non-invasive, readily administered and well tolerated; hence, they are emerging as first-line treatment for patients. The present invention achieves rapid delivery of PDE5 inhibitors in a manner that provides an increased rate of absorption, which in turn allows for greater flexibility in administration. The invention provides a formulation that provides a faster onset of action with a lower dose when compared to purely gastrointestinally absorbed formulations. Buccal and/or sublingual drug absorption, as will be appreciated by those skilled in the art, avoids the disadvantages encountered with gastrointestinal absorption, e.g., slow absorption, degradation of the PDE5 inhibitor by fluids present in the gastrointestinal tract and/or first-pass inactivation by the liver. Lower doses are achievable by at least partially avoiding the metabolism (and resulting loss of bioavailable PDE5 inhibitor) associated with gastrointestinal absorption.

One advantage of oral absorption is a reduced “food effect.” It is commonly known in the art that meal intake may affect pharmacokinetic parameters, because food may decrease the rate and extent of absorption. This is especially true for fatty foods. The present invention overcomes this potential problem by providing buccal and sublingual absorption opportunities, which may or may not be accompanied by gastrointestinal absorption. Thus oral absorption not only avoids loss of available drug substance through metabolism, but also avoids any potential slowing of systemic absorption base on food ingestion.

A second advantage of the formulations of the present invention is that since absorption occurs in the oral cavity, there is no requirement to swallow. Thus, these formulations do not require that the patient have access to a liquid to assist in swallowing an alternate dosage form such as a tablet or capsule. This feature may be advantageous to those who prefer to administer the PDE5 inhibitor discretely, or without respect to immediate access to a potable liquid. Furthermore, since swallowing is not necessary, patients who may be incapable of taking direction, or who are unconscious, can be effectively dosed. This latter feature may be of particular benefit in treating patients for serious cardiovascular conditions, for example, those who may have suffered angina or a stroke and are delivered to an emergency room.

The buccal and/or sublingual delivery of a therapeutically effective amount of a PDE5 inhibitor should result in a higher area under the curve (“AUC”) than would be achieved by similar doses of a solely gastrointestinally absorbed agent. Conversely, orally absorbed dosage forms may allow lower dosages of drug substance to be administered to attain a similar AUC. Lower dosages of PDE5 inhibitors may be advantageous in avoiding some of the reported adverse events, such as headache, blue halo effect and blindness. Optionally, the method may further encompass an extended release formulation to obtain sustained blood levels and activity of the PDE5 or contain a second pharmaceutical agent.

PDE5 inhibitors may be one of the cGMP-specific forms. Examples of suitable PDE5 inhibitors include, but are not limited to, zaprinast, MY5445, dipyridamole, vardenafil, sildenafil, and tadalafil. Other phosphodiesterase type 5 inhibitors include those disclosed in U.S. Pat. No. 6,548,490; U.S. publication No. 2003/0139384; and PCT Publication Nos. WO 94/28902 and WO 96/16644, hereby incorporated by reference.

An example of a particularly preferred PDE5 inhibitor is SCH446132, which is disclosed in U.S. Pat. No. 6,821,978, and is currently in development by Schering Corp. The chemical structure of SCH446132 is as follows:

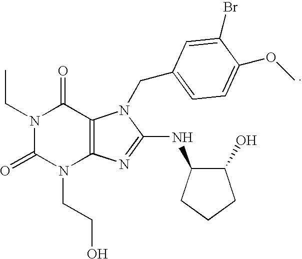

Other suitable PDE5 inhibitors include, but are not limited to, at least one of alprostadil, papavaerine , pentoxifylline, phentolamine, or yohimbine hydrochloride. Preferred PDE5 inhibitors include, but are not limited to, SHC446132, sildenafil citrate, tadalafil, vardenafil, avanafil and udenafil. More preferably, the PDE5 inhibitor is SCH446132.

The PDE5 inhibitor may be administered, if desired, in the form of salts esters, amides, prodrugs, derivatives, and the like, provided the salt, ester, amide, prodrug or derivative is pharmacologically suitable, i.e., effective in the present method. Salts, esters, amides, prodrugs and other derivatives of the active agents may be prepared using standard procedures known to those skilled in the art of synthetic organic chemistry and described, for example, by J. March, Advanced Organic Chemistry; Reactions, Mechanisms and Structure, 4th Ed. (New York: Wiley-Interscience, 1992). For example, acid addition salts are prepared from the free base using conventional methodology, and involve reaction with a suitable acid. Generally, the base form of the drug is dissolved in a polar organic solvent such as methanol or ethanol and the acid is added thereto. The resulting salt either precipitates or may be brought out of solution by addition of a less polar solvent. Suitable acids for preparing acid addition salts include organic acids and inorganic acids. Organic acids include, but are not limited to, acetic acid, propionic acid, glycolic acid, pyruvic acid, oxalic acid, malic acid, malonic acid, succinic acid, maleic acid, fumaric acid tartaric acid, citric acid, benzoic acid, cinnamic acid, mandelic acid, methanesulfonic acid, ethanesulfonic acid, p-toluenesulfonic acid, or salicylic acid. Inorganic acids include, but are not limited to, hydrochloric acid, hydrobromic acid, sulfuric acid, nitric acid, or phosphoric acid.

An acid addition salt may be reconverted to the free base by treatment with a suitable base. Particularly preferred acid addition salts of PDE5 inhibitors are halide salts. Halide salts may be prepared using hydrochloric or hydrobromic acids. Conversely, basic salts of acid moieties which may be present on a phosphodiesterase inhibitor molecule are prepared in a similar manner using a pharmaceutically acceptable base. Bases include, but are not limited to, sodium hydroxide, potassium hydroxide, ammonium hydroxide, calcium hydroxide, or trimethylamine.

Preparation of esters involves functionalization of hydroxyl and/or carboxyl groups which may be present within the molecular structure of the drug. The esters are typically acyl-substituted derivatives of free alcohol groups, i.e., moieties which are derived from carboxylic acids of the formula RCOOH where R is alkyl, and preferably is lower alkyl. Esters can be reconverted to the free acids, if desired, by using conventional hydrogenolysis or hydrolysis procedures. Amides and prodrugs may also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety, which results in a compound that is therapeutically inactive until modified by an individual's metabolic system.

The invention encompasses pharmaceutical formulations comprising at least one PDE5 inhibitor and at least one orally disintegrating carrier, wherein the pharmaceutical formulation disintegrates in about 0.5 to about 120 seconds and/or a therapeutically effective amount of the PDE5 inhibitor is absorbed into the bloodstream within about 1 to about 5 minutes. Preferably, a therapeutically effective amount of the PDE5 inhibitor is absorbed into the bloodstream within about 3 minutes. Some embodiments encompass pharmaceutical formulations comprising at least one PDE5 inhibitor and at least one orally disintegrating carrier, wherein the PDE5 inhibitor achieves a Cₘₐₓ of about 5 μg/L to about 60 μg/L in about 5 minutes to about 10 minutes. In some embodiments, the formulation provides an AUC of PDE5 inhibitor of about 10 μgh/L to about 200 μgh/L.

Optionally, the formulation may contain one or more second pharmaceutical agents, e.g., a dopaminergic drug, a smooth muscle relaxant, a vasoactive drug, or an additive.

The invention encompasses administration of any type of formulation or dosage unit suitable for application to the mucosal tissue. The formulation may be administered in a solid dosage form to be placed on the tongue (lingual formulations), or under the tongue (sublingual formulations), or applied to the buccal mucosa (buccal formulations), or sprayed into the mouth or under the tongue (oral mist). In some embodiments, the formulations comprise a dosage form for application to the sublingual mucosa and a carrier suitable for sublingual drug delivery of the PDE5 inhibitor. Lingual formulations deliver the PDE5 inhibitor by stimulating saliva generation, which enhances disintegration of the formulation, allowing for buccal and/or sublingual absorption. In some embodiments, the formulations comprise a dosage form suitable for forming a suspension of undissolved PDE5 inhibitor particles in saliva, which can then be swallowed, allowing for gastrointestinal absorption and sustained or extended absorption of the PDE5 inhibitor.

The amount of PDE5 inhibitor administered and the dosing regimen used, will depend on the particular drug selected, the age and general condition of the subject being treated, the severity of the subject's condition, and the judgment of the prescribing physician. Thus, because of patient-to-patient variability, dosages are a guideline only and the physician may adjust doses of the compounds to achieve the level of effective treatment that the physician considers appropriate for the patient. In considering the degree of treatment desired the physician must balance a variety of factors such as the age of the patient and the presence of other diseases or conditions (e.g. cardiovascular disease).

A typical daily dose of PDE5 inhibitor to be administered for at least partial transmucosal, i.e., buccal or sublingual, absorption is generally about 0.5 mg to about 100 mg. In some embodiments, the PDE5 inhibitor is present in an amount of about 0.5 mg to about 15 mg, In some embodiments, the PDE5 inhibitor is present in an amount of about 0.5 mg to about 5 mg. In some embodiments, the PDE5 inhibitor is present in an amount of about 0.5 mg to about 3 mg. Depending on the half-life of the PDE5 inhibitor and the availability via the chosen route of administration, the dosing regimen can be modulated in order to achieve satisfactory therapeutic results. Formulations intended to effect both transmucosal and gastrointestinal absorption may encompass higher doses of the PDE5 inhibitor.

The dosage unit will generally contain from approximately 1% to about 60% by weight of at least one PDE5 inhibitor, preferably the PDE5 inhibitor is present in an amount of about 1% to about 30% by weight of the formulation. The above dosages are exemplary of the average case, but there can be individual instances in which higher or lower dosage ranges may be merited, and such are within the scope of the invention.

The orally disintegrating carrier may be a bioerodible (hydrolyzable) polymeric carrier that optionally may also serve to adhere the dosage form to the buccal and/or sublingual mucosa.

In some embodiments, the orally disintegrating carrier of the invention is a carrier capable of forming a gel in the form of a strip. The orally disintegrating carrier should be capable of disintegrating in about 0.5 second to 120 seconds from contact with a surface in the oral cavity. Preferably, the orally disintegrating carrier is capable of disintegrating in about 0.5 second to about 50 seconds. More preferably, the orally disintegrating carrier is capable of disintegrating in less than about 5 seconds.

The orally disintegrating carrier can be any such carrier, so long as the desired drug dissolution profile is not compromised, and the carrier is compatible with the PDE5 inhibitor to be administered and any other component that may be present in the dosage unit. Generally, the orally disintegrating carrier may comprise hydrophilic (water-soluble and water-swellable) polymers that may adhere to a wet surface in the oral cavity. Polymeric carriers include, but are not limited to, acrylic acid polymers; hydrolyzed polyvinylalcohol; polyethylene oxides; polyacrylates; vinyl polymers; polyvinylpyrrolidone; dextran; guar gum; pectins; starches; or cellulosic polymers.

Acrylic polymers include, but are not limited to, polymers known as “carbomers” (e.g., Carbopol®, from B.F. Goodrich). Polyethylene oxides include, but are not limited to, Sentry Polyox® water soluble resins (available from Union Carbide). Polyacrylates include Eudragit® (available from Rohm). Cellulosic polymers include, but are not limited to, hydroxypropyl methylcellulose (e.g., Methocel® from the Dow Chemical Company); hydroxypropyl cellulose (e.g., Klucel® from Dow); hydroxypropyl cellulose ethers (e.g., as disclosed in U.S. Pat. No. 4,704,285, hereby incorporated by reference); hydroxyethyl cellulose; carboxymethyl cellulose; sodium carboxymethyl cellulose; methyl cellulose; ethyl cellulose; cellulose acetate phthalate; cellulose acetate butyrate; microcrystalline cellulose; and the like. Conventional nontoxic solid carters include, but are not limited to, at least one of pharmaceutical grades of mannitol, lactose, starch, magnesium stearate, sodium saccharin, talc, glucose, sucrose, or magnesium carbonate.

Depending on the particular PDE5 inhibitor administered, it may be desirable to incorporate a permeation enhancer in the formulation in order to increase the rate at which the PDE5 inhibitor permeates through the mucosal tissue to which it is applied, e.g., the buccal mucosa, lingual, or sublingual mucosa. These permeation enhancers also are referred to as accelerants, adjuvants, and absorption promoters, and are collectively referred to herein as “permeation enhancers.” The permeation enhancer includes those compounds with diverse mechanisms of action including those which have the function of improving the solubility and diffusibility of the drug, and those which improve percutaneous absorption by changing the ability of the stratum corneum to retain moisture, softening the skin, improving the skin's permeability, acting as penetration assistants, or changing the state of the skin such as the boundary layer.

Suitable permeation enhancers include, but are not limited to, dimethylsulfoxide (“DMSO”), dimethyl formamide (“DMF”), N,N-dimethylacetamlide (“DMA”), decylmethylsulfoxide (“C₁₀ MSO”), polyethylene glycol monolaurate (“PEGML”), glycerol monolaurate, lecithin, 1 substituted azacycloheptan-2-ones, alcohols, or surfactants. Surfactants include, but are not limited to, Tergitol®, Nonoxynol-9®, and TWEEN-80®. 1-Subtsituted azacycioheptan-2-ones include 1-n-dodecylcyclazacycloheptan-2-one (available under the trademark Azone® from Nelson Research & Development Co., Irvine, Calif.) or SEPA® (available from Macrochem Co., Lexington, Mass.). Other permeation enhancers may be found in U.S. publication No. 2003/1393384, hereby incorporated by reference.

Optionally, the formulations may include at least one enzyme inhibitor effective to inhibit drug-degrading enzymes which may be present at the site of administration. Enzyme inhibiting compounds may be determined by the skilled artisan by reference to the pertinent literature and/or using routine experimental methods.

Optionally other ingredients may be incorporated into the pharmaceutical formulation and/or dosage forms. The additional components include, but are not limited to, at least one of pH buffering agents, disintegrants, diluents, binders, emulsifying agents, lubricants, wetting agents, flavoring agents, colorants, preservatives, and the like. Additional components that may be incorporated into sublingual dosage forms are known, or will be apparent, to those skilled in this art. See, Remington: The Science and Practice of Pharmacy, 20th edition (Lippincott, Williams and Wilkins Publishing), p. 859.

Buffering agents include, but are not limited to, sodium acetate, sorbitan monolaurate, triethanolamine sodium acetate, or triethanolamine oleate

Disintegrants include, but are not limited to, cross-linked polyvinylpyrrolidones (e.g., crospovidone, such as Polyplasdone® XL available from GAF); cross-linked carboxylic methylcelluloses (e.g., croscarmelose, such as Ac-di-sol® available from FMC); alginic acid, calcium silicate, and sodium carboxymethyl starches (e.g., Explotab® available from Edward Medell Co., Inc.); methylcellulose; agar bentonite; alginic acid; calcium carbonate; polyoxyethylene sorbitan fatty acid esters; sodium lauryl sulfate; stearic monoglyceride; or lactose.

Suitable diluents are those which are generally useful in pharmaceutical formulations prepared using compression techniques. Diluents include, but are not limited to, dicalcium phosphate dihydrate (e.g., Di-Tab® available from Stauffer); sugars that have been processed by co-crystallization with dextrin (e.g., co-crystallized sucrose and dextrin such as Di-Pak® available from Amstar); lactose; calcium phosphate; cellulose; kaolin; mannitol; sodium chloride; dry starch; powdered sugar; and the like.

Binders are those compounds that enhance adhesion. Binders include, but are not limited to, water, ethanol, polyvinylpyrrolidone, starch, gelatin, or sugars. Sugars include sucrose, dextrose, molasses, and lactose. Lubricants include, but are not limited to, stearic acid, polyethylene glycol, or stearates, such as magnesium stearate. Wetting agents include, but are not limited to, glycerin, starches, and the like.

Conventional flavoring agents may be used, such as those described in Remington: The Science and Practice of Pharmacy, 20^{th} Ed. (Lippincott, Williams and Wilkins Publishing,), which is incorporated herein by reference. The pharmaceutical compositions of the invention generally contain from about 0 to 2% by weight of a flavoring agent.

Conventional colorants such as dyes and/or pigments may also be used, such as those described in the Handbook of Pharmaceutical Excipients, by the American Pharmaceutical Association & the Pharmaceutical Society of Great Britain, pp. 81-90 (1986), which is incorporated herein by reference. The pharmaceutical compositions of the invention generally contain from about 0 to 2% by weight of colorants.

Dosage Forms

In certain embodiments, the formulations of the invention are in dosage forms for direct application to the buccal, lingual area, or sublingual area to achieve rapid onset. When lingually applied (on the tongue), the dosage form stimulates saliva production, thus enhancing rapid disintegration of the dosage form and dissolution of the PDE5 inhibitor. When applied sublingually, the dosage form is applied directly to the absorptive membrane on the underside of the tongue. For example, the dosage form may be in the form of a strip, oral mist, granulated particles, gum, lyophilized wafer/tablet, lozenge, pill, tablet, rapidly disintegrating tablet, troche, and the like that has the disintegration properties discussed above. Preferred dosage forms include, but are not limited to, strips, oral mists, rapidly disintegrating tablets, lyophilized wafer/tablet, and granulated particles.

In some embodiments incorporating granulated particles, the particles have median sizes of about 50 to about 500 microns. In some embodiments, the median particle size is between about 100 and about 200 microns. The granulated particles may be formed by any of a variety of processes including spheronization, milling, de-agglomeration, precipitation, and/or crystallization. The use of granulated particles in solid dosage forms is taught in U.S. Pat. No. 5,178,878, which is incorporate in its entirety herein by reference.

When in strip form, the dosage form should disintegrate and disperse rapidly and provide for high bioavailability of the PDE5 inhibitor. The strips may be applied to either or both of the top side or bottom side of the tongue. Strips to be applied under the tongue may be shaped with curved edges in order that the dosage unit may fit comfortably and precisely in the suiblingual cavity. In some embodiments, the dosage form is a rapidly disintegrating tablets such as a formulation that disintegrates in the mouth within seconds of placement on the tongue, allowing rapid release of the PDE5 inhibitor. Effervescent agents, such as those taught in U.S. Pat. No. 5,178,878, may be incorporated to speed disintegration of the dosage form in the oral cavity.

The sublingual dosage forms of the present invention can be manufactured using conventional processes. The sublingual dosage unit is fabricated to disintegrate rapidly. The time period for complete disintegration of the dosage unit is typically in the range as described above. Actual methods of preparing such dosage forms are known, or will be apparent, to those skilled in this art. See, Remington: The Science and Practice of Pharmacy, 20^{th} Ed., (Lippincott, Williams and Wilkins Publishing).

Another dosage fonn of the present invention is an oral mist, such as an aerosol. The oral mist can be administered lingually, buccally, or sublingually. The oral mist can be conveniently delivered in the form of a dry powder inhaler or an aerosol spray presentation from a pressurized container, non-pressurized dispenser, pump, spray or nebulizer with the use of a suitable propellant. Propellants include, but are not limited to, dichlorodifluoromethane, trichlorofluoromethane, dichlorotetrafluoroethane, hydrofluoroalkanes, carbon dioxide, or inert gases. Hydrofluoroalkanes include 1,1,1,2-tetrafluoroethane or 1,1,1,2,3,3,3-heptafluoropropane. Inert gasses include nitrogen or argon. In the case of a pressurized aerosol, the dosage unit may be determined by providing a valve to deliver a metered amount. The pressurized container, pump, spray, or nebulizer may contain a solution or suspension of the PDE5 inhibitor, e.g., using a mixture of ethanol and the propellant as the solvent, which may additionally contain a lubricant, e.g., sorbitan trioleate. Non-pressurized dispensers include those in which the patient administers the drug product in a form suitable for at least one of the buccal, sublingual, or gastrointestinal absorption. Capsules or cartridges for use in an inhaler or insufflactor may be formulated to contain a powder mix of the PDE5 inhibitor and a suitable powder base such as lactose or starch. Aerosol or dry powder formulations are preferably arranged so that each metered dose or “puff” contains the desired amount of PDE5 inhibitor as discussed above.

In those embodiments in which the PDE5 inhibitor is formulated for delivery via an atomizer, formulations may contain additional ingredients such as solubilisers, emulsifiers, or suspending agents.

Optionally, the formulation may additionally contain an extended release component for gastrointestinal absorption for sustained duration of action. The extended release component is intended to provide the PDE5 inhibitor and/or second pharmaceutical agent over a longer period of time. The extended release component comprises at least one PDE5 inhibitor and a non-orally disintegrating carrier, allowing a portion of the PDE5 inhibitor and/or second pharmaceutical agent to be swallowed for gastrointestinal absorption. The extended release component of the formulation may comprise the core of a tablet whose outer layer is comprised of a rapidly disintegrating component. In other embodiments, the extended release component comprises slowly dissolving particles. For example, in some embodiments, a plurality of slowly dissolving particles is coated, individually or collectively, with an immediate release formulation. In other embodiments, the pharmaceutical formulation is formed as a strip comprising extended release granulated particles in a matrix containing the rapidly disintegrating and dissolving component.

Pharmacokinetic Profile

The present invention overcomes the problems of the prior art administrations by providing a formulation for delivering PDE5 inhibitors quickly and achieving rapid bioavailability. Not to be limited by theory, it is believed that buccal and/or sublingual administration of the PDE5 inhibitor can achieve more advantageous pharmacokinetic parameters than oral dosages solely absorbed through the gastrointestinal tract. Because of the route of administration, the formulations and methods of the invention achieve a more rapid onset of action and similar AUCs using lesser dosed amounts of the PDE5 inhibitor than the amounts required in conventional solid oral dosage forms.

Moreover, the pharmacokinetic profile of the formulations of the invention is believed to be superior to the prior art formulations in that the time to reach effective blood levels is believed to be decreased, while the AUC is believed to be equal or similar to gastrointestinally absorbed drugs administered in much higher doses. The rapid delivery of the active agent is believed to allow for a rapid achievement of therapeutic levels and a faster Tₘₐₓ.

For example, it is believed that the pharmaceutical formulations are capable of disintegrating or dispersing in the mouth in about 1 to about 10 seconds and the PDE5 inhibitor is absorbed in the bloodstream such that therapeutic levels are attained within about 1 to about 5 minutes. Preferably, the PDE5 inhibitor will reach therapeutic levels within 3 minutes or less. The invention encompasses pharmaceutical formulations wherein the PDE5 inhibitor is believed to achieve a Cₘₐₓ of about 5 μg/L to about 60 μg/L in about 5 minutes to about 10 minutes and an AUC of about 10 μgh/L to about 200 μgh/L.

In some embodiments, the PDE5 inhibitor is believed to achieve a Cₘₐₓ of about 200 μg/L to about 400 μg/L in about 5 minutes to about 10 minutes and an AUC of about 4000 μgh/L to about 9000 μgh/L. Extended release versions of this embodiment are believed to achieve an AUC of about 8000 μgh/L to about 15,000 μgh/L.

The formulations of the invention are believed to have a systemic effect over a period from about 2 minutes to about 24 hours. Preferably, the systemic effect is believed to be from about 2 minutes to about 12 hours. Typically, the time for onset is believed to be about 1 minute to about 20 minutes. Preferably, the onset time is believed to be less than about 10 minutes. More preferably, the onset time is believed to be about 3 minutes.

Diseases to be Treated

The formulations of the invention may be used to treat a disease state treatable with a PDE5 inhibitor (“a PDE5-treatable condition”). The biochemical, physiological, and clinical effects of PDE5 inhibitors suggest their utility in a variety of diseases in which modulation of smooth muscle, renal, hemostatic, inflammatory, and/or endocrine function is desirable. Diseases treated by PDE5 inhibitors include, but are not limited to, erectile dysfunction, premature ejaculation, female sexual dysfunction, cardiovascular, cerebral stroke, congestive heart failure, cerebrovascular conditions, ischemic heart disease, pulmonary arterial hypertension, acute respiratory distress syndrome, benign prostatic hypertrophy, atherosclerosis, autoimmune diseases, overactive bladder, bladder outlet obstruction, incontinence, cachexia, cancer, diabetes, endarterectomy, diseases characterized by disorders of gut motility, dysmenorrhoea, elevated intra-occular pressure, glaucoma, glomerular renal insufficiency, hyperglycemia, hypertension, impaired glucose tolerance, inflammatory diseases, insulin resistance syndrome, intestinal motility, macular degeneration, nephritis, optic neuropathy, osteoporosis, peripheral arteial disease, polycystic ovarian syndrome, renal failure, respiratory tract disorders, thrombocythemia, tubular interstitial diseases, and urologic disorders. Urological disorders include female and male sexual dysfunctions.

Allergic disorders associated with atopy include, but are not limited to, urticaria, eczema, or rhinitis.

Cardiovascular diseases include, but are not limited to, atherosclerosis, restenosis, hypertension, acute coronary syndrome, angina pectoris, arrhythmia, a cardiovascular disease associated with hormone replacement therapy, cerebral infarction, cerebral ischemia, conditions of reduced blood vessel patency (e.g., postpercutaneous transluminal coronary or carotid angioplasty, or post-bypass surgery graft stenosis), deep vein thrombosis, disseminated intravascular coagulation syndrome, heart disease, heart failure, migraine, myocardial infarction, peripheral vascular disease, Raynaud's disease, renal ischemia, renal vascular homeostasis, thrombotic or thromboembolytic stroke, venous thromboembolism, pulmonary arterial hypertension, congestive heart failure, myocardial infarction and angina, and prevention of any such cardiovascular condition or event subsequent to a first cardiovascular event (i.e., “secondary prevention”).

Diseases characterized by disorders of gut motility include, but are not limited to, irritable bowel syndrome, diabetic gastroparesis and dyspepsia.

Female sexual dysfunction (FSD) includes, but is not limited to, clitoral dysfunction, female hypoactive sexual desire disorder, female sexual arousal disorder (FSAD), female sexual pain disorder, and female sexual orgasmic dysfunction (FSOD)

Respiratory tract disorders include, but are not limited to, acute respiratory failure, allergic asthma, allergic rhinitis, bronchitis, chronic asthma, reversible airway obstruction, and allergic disorders associated with atopy (such as urticaria, eczema, or rinitis).

Other medical conditions for which a PDE5 inhibitor is indicated, and for which treatment with the formulations of the present invention may be useful include, but are not limited to, pre-eclampsia, Kawasaki's syndrome, nitrate tolerance, multiple sclerosis, diabetic nephropathy, neuropathy including autonomic and peripheral neuropathy and in particular diabetic neuropathy and symptoms thereof (e.g., gastroparesis, peripheral diabetic neuropathy), Alzheinmer's disease, psoriasis, skin necrosis, metastasis, baldness, nutcracker oesophagus, anal fissure, hemorrhoids, insulin resistance syndrome, hypoxic vasoconstriction as well as the stabilization of blood pressure during haemodialysis.

Preferably, the diseases treated using the formulations of the invention include erectile dysfunction, pulmonary arterial hypertension, congestive heart failure, benign prostatic hypertrophy, myocardial infarction and angina.

Combination Therapy

It is understood that other combinations may be undertaken while remaining within the scope of the invention. While one or more of the PDE5 inhibitors may be used in an application of monotherapy to treat PDE5-treatable conditions, the formulations of the invention may be used also in combination therapy. In some embodiments, the formulations of the invention are combined with one or more second pharmaceutical agents that are useful for treating other types of disorders, symptoms, or diseases, For example, the pharmaceutical formulation may be administered with a second pharmaceutical agent that may cause a PDE5-treatable condition as a side effect. One example of such a second pharmaceutical agent is SRRIs, which are useful for treating depression, but which can have various forms of sexual dysfunction as a side effect. SSRIs include, but are not limited to, paroxetine, fluoxetine, sertraline, fluroxamine, citalopram and escitalopram. Paroxetine is a particularly popular example of an SSRI that might be considered for combination therapy within the scope of the present invention.

Typically, drugs that may cause impotence include, but are not limited to, anti-androgens, anti-anxiety drugs, endoenne, anti-cholinergic drugs, anti-nausea, anti-hypertensives, chemo-therapeutic agents, psychotropics, histamine receptor antagonists, and anti-hyperipipdemics. Endoenne drugs include estrogens, anti-androgens, lutenizing hormone-releasing hormone (LHRH) analogues, and 5 alpha reductase inhibitors. Anti-hypertensive drugs include diuretics, methyldopa, beta blockers, and Ca antagonists. Psychotropic drugs include major tranquilizers, monoamine oxidase (MAO) inhibitors, selective serotonin reuptake inhibitors, and tricyclo anti-depressants.

The present invention also encompasses combination therapy with a second pharmaceutical agent which is being administered to treat a disease or condition which has, as a symptom or complication, a PDE5-treatable condition. Thus, a PDE5 inhibitor may be administered along with a second pharmaceutical agent intended to treat a condition that has erectile dysfunction as a symptom. Diseases that may cause sexual dysfunction include, but are not limited to, craniopharyngioma, diabetes, epilepsy, hypogonadism, hypertension, ischemic heart disease, multiple sclerosis, and/or peripheral vascular disease. Thus, for example, combination therapies comprising co-administration of an anti-epileptic and a PDE5 inhibitor are within the scope of the present invention.

Also within the scope of the present invention are methods of treating a patient in need of such treatment by administering a pharmaceutical formulation as herein described. Such patients include those with a PDE5treatable condition, those with a condition treatable by a second pharmaceutical agent known to cause a PDE5-treatable condition, and those with a condition which has as a known symptom or secondary effect, a PDE5-treatable condition.

Administration of the PDE5 inhibitor and second pharmaceutical agent in combination typically is carried out over a defined time period. For example, the combination may be administered simultaneously or within minutes, hours, days, or weeks depending upon the combination selected.

Combination therapy is intended to embrace administration of the PDE5 inhibitor and second pharmaceutical agent either in a substantially simultaneous manner or a sequential manner. For example, substantially simultaneous administration can be accomplished by administering to a subject a single strip having a fixed ratio of each of the PDE5 inhibitor and second pharmaceutical agent or in discrete capsules, tablets, or strips for each of the agents. The PDE5 inhibitor and the second pharmaceutical agent may be included in a single dosage form, or the two may be separately administered, each in its respective dosage form.

As used herein, the term “erectile dysfunction” is intended to include any and all types of erectile dysfunction, including: vasculogenic, neurogenic, endocrinologic and psychogenic impotence, Peyronie's syndrome, premature ejaculation, and any other condition, disease, or disorder, regardless of cause or origin, which interferes with at least one of the three phases of human sexual response, i.e., desire, excitement and orgasm. As used herein, the term “impotence” is used here in its broadest sense to indicate a periodic or consistent inability to achieve or sustain an erection of sufficient rigidity for sexual intercourse. See, U.S. Pat. No. 5,242,391; U.S. patent publication No. 2003/0139384.

As used herein, the term “perneation enhancer” refers to an agent that accelerates the delivery of the drug through the mucosa.

As used herein, the terms “phosphodiesterase Type 5”, “phosphodiesterase Type V”, “PDE5” and “PDE V” are used interchangeably.

As used herein, the term “orally” is understood to refer to the oral cavity, i.e., the mouth, or to any of the bodily surfaces contained therein. Thus, an “orally disintegrating” formulation or carrier is one that disintegrates in the mouth, whether lingually, sublingually, or buccally.

As used herein, the teen “orally disintegrating carrier” means a carrier capable of dissolving, dispersing or disintegrating, within the oral cavity, including lingually or sublingually, as well as on the wvalls of the mouth once placed in the mouth and coming into contact with the mucosal tissue of the tongue, cheek, or mouth.

As used herein, the term “nonorally disintegrating carrer” means a carrier capable of delivering at least a portion of the PDE5 inhibitor to the gastrointestinal tract for absorption there.

As used herein, the terms “treating” and “treatment” refer to at least one of reduction in severity and/or frequency of symptoms, elimination of symptoms and/or underlying cause, prevention of the occurrence of symptoms and/or their underlying cause, or improvement or remediation of damage. For example, the present method of “treating” erectile dysfunction, as the term is used herein, thus encompasses both prevention of the disorder in a predisposed individual and treatment of the disorder in a clinically symptomatic individual. As used herein, the term “transmucosai” drug delivery means administration of a drug to the mucosal surface of an individual so that the drug passes through the mucosal tissue and into the individual's blood stream. A preferred form of transmucosal drug delivery herein is “buccal” or “transhuccal” drug delivery, which refer to delivery of a drug by passage of the drug through an individual's buccal mucosa and into the bloodstream. Another preferred form of transmucosal drug delivery herein is “sublingual” or “transublingual” drug delivery, which refer to delivery of a drug by passage of the drug through an individual's sublingual mucosa and into the bloodstream.

As used herein, the term “lingual strip” means a narrow piece of material to be placed on the superior or lateral sides of the tongue.

As used herein, the term “sublingual strip” means a narrow piece of material to be placed below the tongue or between the tongue and the bottom of the mouth.

As used herein, the term “oral mist” means a pharmaceutical formulation formulated as a liquid or particulate matter in air, gas, or vapor in the form of a fine mist for therapeutic purposes. The oral mist may be packaged under pressure and contain therapeutically active ingredients intended for topical application, inhalation, or administered by absorption through the mucosal tissue of the mouth.

As used herein, the term “rapidly disintegrating tablet” means a tablet that disintegrates within about 1 second to about 10 seconds once placed in the mouth and coming into contact with the mucosal tissue of the tongue, cheek, or mouth

As used herein, the term “lyophilized wafer” means a thin dosage form used to include the PDE5 inhibitor alone or in combination with the second pharmaceutical agent or sustained release PDE5 inhibitor component, which dosage form has been fabricated by a freeze drying process. The wafer may be moistened and folded over the PDE5 inhibitor and/or second pharmaceutical agent to mask the taste.

As used herein, the term “granulated particles” means a pharmaceutical formulation in the form of particles or spheres.

As used herein, the term “extended release component” means a pharmaceutical formulation designed to gradually and continually release amounts of at least one PDE5 inhibitor and/or second pharmaceutical agent to maintain a level of therapeutic or prophylactic effect over an extended period of time. In some embodiments, in order to maintain a constant level of drug in the body, the drug is released from the dosage form at a rate that will replace the amount of drug being metabolized and excreted from the body.

As used herein, the term “effective” or “therapeutically effective” amount of a drug or pharmacologically active agent means an amount that is sufficient to provide the desired therapeutic effect, e.g., treatment of erectile dysfunction.

As used herein, the term “SSRI” means selective serotonin reuptake inhibitor.

As used herein, the term Cₘₐₓ means the maximum value of PDE5 inhibitor concentration in the patient's blood attained after administration of the pharmaceutical formulation.

The term “about”, when used herein as a modifier of a Cₘₐₓ value or an AUC value, means within a range of 80-125% of the relevant value. Thus, for example, an AUC value of “about 10 μgh/L” means an AUC value in the range of 8-12.5 μgh/L.

While the present invention is described with respect to particular examples and preferred embodiments, it is understood that the present invention is not limited to these examples and embodiments. The present invention, as claimed, therefore includes variations from the particular examples and preferred embodiments described herein, as will be apparent to one of skill in the art.

### EXAMPLES

The following examples are prophetic and illustrative of various embodiments within the scope of the present invention.

### Example 1

A sublingual tablet is prepared by blending sildenafil citrate (1.0 g), mannitol (1.0 g), microcrystalline cellulose (2 g), and magnesium stearate (10 mg,) in a suitable mixer and then compressing the mixture into sublingual tablets. Each sublingual tablet contains 10 mg of sildenafil citrate.

### Example 2

A sublingual tablet is prepared by blending SCH446132 (0.5 g), mannitol (1.0 g), microcrystalline cellulose (2.0 g), and magnesium stearate (10 mg) in a suitable mixer and then compressing the mixture into sublingual tablets. Each sublingual tablet contains 5 mg of SCH446132.

### Example 3

A lingual/sublingual wafer is prepared by mixing SCH446132 (10 g) in a solution containing gelatin and mannitol. The liquid mixture is filled into blister trays and lyophilized. Each lyophilized wafer contains 5 mg of SCH446132.

### Example 4

Lingual/sublingual dissolving granules are prepared by mixing vardenafil hydrochloride (10 g) with sucrose (90 g). The mass is granulated using a solution of water and PVP and dried. The dried granules are weighed into individual sachets in unit dose amounts. Each sachet contains 3 mg of vardenafil

### Example 5

A lingual/sublingual spray is prepared by mixing sildenafil citrate (5 g) in water (100 mL) containing ethanol (10 mL). The solution is filled into bottles with fixed dose spray pump and valve assembly. Each spray delivers 5 mg of sildenafil citrate.

### Example 6

A lingual/sublingual spray is prepared by mixing SCH446132 (5 g) in water (100 mL) containing ethanol (10 mL). The solution is filled into bottles with fixed dose spray pump and valve assembly. Each spray delivers 3 mg of SCH446132.

### Example 7

A lingual/sublingual film strip is prepared by mixing sildenafil citrate (10 g) in molten gelatin (90 g). The mixture is cast into circular or appropriately shaped individual films and packed as individual units. Each strip contains 5 mg of sildenafil citrate.

### Example 8

An immediate release/extended release wafer is prepared by mixing SCH446132 (10 g ) in a solution containing gelatin and mannitol. An additional 10 g of SCH446132 is extruded and spheronized with microcrystalline cellulose (90 g) and dried to create granules/spheres. The SCH446132 granulation is coated using a polyacrylate polymer and suspended in the previously prepared solution. The suspension is filled into blister trays and lyophilized. Each lyophilized wafer contains up to 20 mg of SCH446132.

### Example 9

An immediate release/extended release film strip is prepared by mixing vardenafil hydrochloride (10 g) in molten gelatin (90 g). An additional 10 g of vardenafil hydrochloride is extruded and spheronized with microcrystalline cellulose (90 g) and dried to create granules/spheres. The vardenafil granulation is coated using a polyacrylate polymer and suspended in the previously prepared solution and the suspension cast into circular or appropriately shaped individual films and packed as individual units. Each strip contains up to 20 mg of vardenafil.

1. A pharmaceutical formulation comprising a rapid release component comprising vardenafil and an orally disintegrating carrier, wherein the rapid release component results in a therapeutically effective blood concentration of the vardenafil in about 1 minute to about 20 minutes.

2-3. (canceled)

4. The pharmaceutical formulation according to claim 1, wherein the rapid release component disintegrates in less than about 5 seconds.

5. The pharmaceutical formulation according to claim 1, wherein the pharmaceutical formulation is in a dosage form selected from the group consisting of lingual strips, sublingual strips, oral mists, rapidly disintegrating tablets, lyophilized wafers, granulated particles and gums.

6. The pharmaceutical formulation according to claim 1, wherein the pharmaceutical formulation is in the form of a lingual strip.

7. The pharmaceutical formulation according to claim 1, wherein the pharmaceutical formulation is in the form of a rapidly disintegrating tablet.

8. The pharmaceutical formulation according to claim 1, further comprising an extended release component comprising vardenafil and a non-orally disintegrating carrier.

9. The pharmaceutical formulation according to claim 8, wherein the pharmaceutical formulation is in the form of a tablet comprising a core comprising the extended release component and a coating comprising the rapid release component.

10. The pharmaceutical formulation according to claim 8, wherein the pharmaceutical formulation is in the form of a strip and the extended release component comprises granulated particles.

11. The pharmaceutical formulation according to claim 1, wherein the pharmaceutical formulation further comprises at least one second pharmaceutical agent.

12. The pharmaceutical formulation according to claim 11, wherein the second pharmaceutical agent is selected from pharmaceutical agents known to cause a PDE5-treatable condition.

13. The formulation of claim 12, wherein said PDE5-treatable condition is erectile dysfunction or premature ejaculaton.

14. The pharmaceutical formulation according to claim 11, wherein the second pharmaceutical agent is known to treat craniopharyngioma, diabetes, epilepsy, hypogonadism, hypertension, ischemic heart disease, multiple sclerosis, or peripheral vascular disease.

15. The pharmaceutical formulation according to claim 1, which formulation results in a Cₘₐₓ of about 5 μg/L to about 60 μg/L in about 5 minutes to about 10 minutes.

16. The pharmaceutical formulation according to claim 1, which formulation results in an AUC of about 10 μgh/L to about 200 μgh/L.

17. The pharmaceutical formulation according to claim 8, which formulation results in an AUC of about 20 μgh/L to about 400 μgh/L.

18. The pharmaceutical formulation according to claim 1, further comprising at least one permeation enhancer selected from the group consisting of DMSO, DMF, DMA, C₁₀ MSO, PEGML, glycerol monolaurate, lecithin, 1-substituted azacycloheptan-2-ones, alcohols, and surfactants.

19. The pharmaceutical formulation according to claim 1, wherein the rapid release component disintegrates within about 1 second to about 10 seconds.

20. A pharmaceutical formulation comprising vardenafil in a lyophilized lingual/sublingual wafer.

21. A pharmaceutical formulation comprising vardenafil and an effervescent agent.

22. A pharmaceutical formulation comprising vardenafil in a spray mist.
